(19) United States
(12) Patent Application Publication
(10) Pub. No.: US 2024/0382598 A1
(43) Pub. Date: Nov. 21, 2024

(54) LIPID FORMULATIONS FOR THE PREPARATION OF HEALTH INGREDIENT-CONTAINING HOT DRINKS

(71) Applicant: Liposoma Health B.V., Groningen (NL)

(72) Inventors: Josbert Maarten Metselaar, Groningen (NL); Grzegorz Pawlik, Groningen (NL); Martino Ambrosini, Groningen (NL); Sandra Nurttila, Groningen (NL)

(21) Appl. No.: 18/690,807

(22) PCT Filed: Sep. 9, 2022
(86) PCT No.: PCT/NL22/50511

§ 371 Date: Mar. 11, 2024

(30) Foreign Application Priority Data
Sep. 10, 2021 (NL) 2029172

(51) Int. Cl. A61K 47/24; A61J 1/10; A61J 1/14; A61K 31/164; A61K 31/352; A61K 31/375; A61K 36/185; A61K 47/02; A61K 47/10; A61K 47/14

(52) U.S. Cl. CPC A61K 47/24; A61J 1/10; A61J 1/1468; A61K 31/164; A61K 31/352; A61K 31/375; A61K 36/185; A61K 36/5777; A61K 47/02; A61K 47/10; A61K 47/14

(57) ABSTRACT

The invention relates to mixtures comprising one or more phospholipids, wherein at least 2 wt % of the phospholipids bears a net negative charge, and/or is deprotonable, whereby the mixture comprises a weak basic salt; one or more oils, butters and/or triglycerides; one or more lipophilic or poorly water-soluble biologically active ingredients; and one or more liquid or solid polyols, sugars, monohydric and/or poly hydric alcohols. The invention further relates to sealed and air- or oxygen-impermeable and liquid-im-permeable containers comprising such mixture and the preparation thereof.

### FIELD OF THE INVENTION

The invention relates to formulations comprising mixtures of poorly water-soluble and/or lipophilic biological active substances with oils, polyols and colloid-forming phospholipids as excipients-which are potentially bioactive also—suitable for filling into a container that protects the mixture from moisture and oxygen during storage, and that can be emptied into and mixed with warm water or other hot aqueous drinks so as to reproducibly prepare a high quality homogeneous fluid comestible product that contains a combination of a phospholipid dispersion and biological actives, with enhanced intestinal uptake of the bioactives after oral administration.

### BACKGROUND OF THE INVENTION

Many lipophilic and/or poorly water soluble biologically active compounds suffer from limited absorption in the gastrointestinal tract after oral administration, which leads to low bioavailability and suboptimal efficacy in the body. It is known that their uptake can be enhanced by formulating them in the form of colloidal oil dispersions in water that can be consumed as a health promoting beverage. Phospholipids (lecithin) are natural lipidic colloid-forming excipients that are capable of creating high quality stable lipid dispersions that can improve the uptake and efficacy of a range of bioactives of different physicochemical nature. Phospholipids can help form liposomes (phospholipid bilayer vesicles) but also micro- or nanoemulsions, as well as (mixed) micelles that can protect and carry a range of delicate bioactives while passing through the hostile gastric milieu on their way to the intestinal environment where they can help increase the bioaccessibility of the bioactives to the enterocytes and improve intestinal uptake.

Unfortunately many biologically actives—and in particular those that are of natural origin—are not only vulnerable to degradation once ingested but are prone to hydrolysis and oxidation when formulated and stored in the form of a comestible aqueous health product. Also phospholipids are sensitive to hydrolysis and oxidation when not properly stored and preserved. Besides antioxidants also significant concentrations of chemical preservatives and/or severe heat treatments are needed to preserve the quality of such products during storage. To simply administer the bioactives and phospholipids as oil formulations, however, is often not feasible due to the lack of solubility of many bioactives in the oil or simply due to the fact that the required dose for health benefit would entail the administration of a large amount of carrier oil.

A promising strategy therefore, which has also been explored in the art, is to formulate the bioactive-containing formulations as concentrated premixes comprising triglyceride oils and glycerol containing phospholipids and other surface active colloid-forming ingredients that can be diluted with water to prepare a beverage shortly before consumption so that the risk of water-induced degradation (e.g. hydrolysis) during storage is minimized. Based on what is known in the art, however, a skilled person would need to formulate significant amounts of (synthetic) surfactants with high hydrophilic-lipophilic balance (HLB) values into such premixes, as for instance described in U.S. Pat. No. 8,187,615 (Friedman, 2012), U.S. Pat. No. 7,393,548 (Friedman, 2008), U.S. Pat. No. 2,463,738 (Bernhart, 1949), and U.S. Pat. No. 6,544,530 (Friedman, 2003). Phospholipids can be regarded in general as lower HLB surfactants and as such are insufficiently able to spontaneously disperse oils in hydrophilic solvents like glycerol.

U.S. Patent application No 2020/0093151 A1 (Nugent, 2020) presents a way to circumvent the need for high HLB surfactants by employing an intricate method of controlled slow addition of a bioactive-containing phospholipid in oil mixture to glycerol within a certain high temperature range while high shear mixing is applied to ensure the formation of a homogenous mixture. The main issue with the cited approach is that both the formulation of the premix as well as the preparation of the eventual aqueous colloidal dispersion after diluting the premix in water requires high-shear homogenization in order to obtain a high quality stable colloidal dispersion that is needed for optimal protection, delivery, bioaccessibility and bioavailability of the incorporated actives. Since the preparation of the lipid dispersion health beverage eventually must be done by the end user, this requires them to have highly specialized equipment readily available in their household and to know how to properly use it.

Therefore, there is a need for concentrated premix formulations of in particular natural oils, phospholipids and polyols that can hold a range of different bioactives, which—in a way that can be handled and accommodated for using the equipment available in a typical household—produces a high quality stable and homogeneous colloidal dispersion that can be consumed as a tasteful health beverage on the spot.

### SUMMARY OF THE INVENTION

It is an object of the present invention to provide an oil-phospholipid-polyol bioactive-containing premix formulation that can be prepared into a phospholipid dispersion for oral intake by simple mixing in warm water. The key element of the invention that allows for this to be achieved is the improved dispersion capacity of the phospholipids in the premix that is obtained and ensured by having a certain fraction of negatively charged phospholipids in the premix, which can be added to the premix and/or be created by treating deprotonable phospholipids present in the premix with weak basic salt.

The invention therefore provides a sealed and air- or oxygen-impermeable and liquid-impermeable container, which encapsulates a mixture comprising:
one or more phospholipids, wherein at least 2 wt % of the phospholipids:
   bears a net negative charge, and/or
   is deprotonable, whereby the mixture comprises a weak basic salt,
one or more oils, butters and/or triglycerides,
one or more lipophilic or poorly water-soluble biologically active ingredients, and
one or more liquid or solid polyols, polyol sugars, natural sugars, monohydric and/or polyhydric alcohols.

The mixture is suitable for mixing into hot water, forming a homogenous phospholipid dispersion, preferably colloidal phospholipid dispersion, without the need for high-sheer homogenization equipment and high HLB synthetic surfactants.

The container is formed of a material which is water- and oxygen-impermeable or has at least one water- and oxygen-impermeable layer.

The container that holds the premix can be designed as airtight sachets with one dosing unit, as a multidose airless dispenser flask or cartridge, or as a multidose airtight jar. Another possibility to hold and process the premix is to encapsulate it in containers that are coffee cup cartridges that are suitable for extraction in coffee machines that are available in most households. In the latter case the cartridge further comprises an inlet port configured to allow a heated and pressurized liquid to be injected into the sealed chamber to form the phospholipid dispersion and an outlet port configured to allow the prepared phospholipid dispersion to exit the sealed chamber. The container may be made of plastic or aluminium or any other airtight material, that in a preferred embodiment is recyclable or biodegradable. Another possibility is to hold the premix in airtight bags that fit into piston-driven chambers that dispense the mix in the required dose in a vending machine configuration.

In another aspect, the invention provides a mixture comprising:
one or more phospholipids, wherein at least 2 wt % of the phospholipids:
   bears a net negative charge, and/or
   is deprotonable, whereby the mixture comprises a weak basic salt,
one or more oils, butters and/or triglycerides,
one or more lipophilic or poorly water-soluble biologically active ingredients, and
one or more liquid or solid polyols, polyol sugars, natural sugars, monohydric and/or polyhydric alcohols.

The mixture is suitable for encapsulating into air- or oxygen-impermeable and liquid-impermeable container/cartridge and for mixing into hot water, forming a homogenous phospholipid dispersion, preferably colloidal phospholipid dispersion, without the need for high-sheer homogenization equipment and high HLB synthetic surfactants.

Said one or more oils and phospholipids, one or more optional additional surfactants and one or more lipophilic and/or lipophilic poorly water-soluble active ingredients may be dissolved and/or dispersed in said one or more liquid monohydric and/or polyhydric alcohols.

Preferably, the mixture is a liquid, viscous liquid.

In another aspect, the invention provides a method for preparing an air- or oxygen-impermeable and liquid-impermeable container according to the invention comprising:
a) mixing one or more phospholipids, wherein at least 2 weight % of the phospholipids is deprotonable and the other lipophilic ingredients with the one or more oils, butters and/or triglycerides,
b) adding the one or more liquid or solid polyols, polyol sugars, natural sugars, monohydric and/or polyhydric alcohols with a weak basic salt dissolved in it to the mixture obtained in step a), whereby two phases are formed,
c) mixing both phases under moderate heating thereby creating and ensuring a sufficiently deprotonated phospholipid fraction,
d) optionally adding other biologically active ingredients that cannot be dissolved in the lipid mixture or the polyol phase beforehand to the mixture obtained in step c),
e) optionally adding and mixing salts, taste enhancers and other food ingredients, such as milk powder, into the mixture obtained in step d) under moderate heating, preferably not exceeding 80° C., and
f) filling an air- or oxygen-impermeable and liquid-impermeable container with the mixture obtained in step e).

### DETAILED DESCRIPTION

The invention pertains to a mixture, preferably encapsulated in a sealed water- and airtight container, which comprises one or more oils, one or more phospholipids, the phospholipids containing a certain fraction of deprotonable components and/or components that bear a net negative charge, optionally added surfactants, and one or more lipophilic and/or poorly water-soluble health ingredients, mixed in monohydric and/or polyhydric alcohol solvent to result in a viscous liquid or a paste. In another embodiment the mixture results in a solid material. Most of the mixture may not yet be present in the form as a colloidal solubilisate but rather as a crude water-free mixture, which—upon exposure to and mixing with warm water (or other warm water-based beverages)—forms a finely dispersed phospholipid dispersion of the lipophilic and/or poorly water-soluble active ingredient(s), which can be served as a health beverage in a cup or a glass. This phospholipid dispersion preferably remains stable without precipitation or phase separation until at least a half hour after preparation at ambient temperature. Within this timeframe the beverage is preferably fully consumed to ensure the bioactive ingredient(s) remain sufficiently, preferably molecularly, dispersed or dissolved in the lipid matrix while passing through the gastrointestinal tract, so that maximal bioavailability and uptake are achieved.

As shown in de examples the formulations to be incorporated in the containers can successfully be made with bioactive substances or mixtures thereof that are known to be lipophilic, poorly water-soluble and/or poorly absorbable by the human body, including bioactive oils, fatty acid esters and amides, cannabinoids, vitamins and provitamins, polyphenols and flavonoids, and essential oil components.

The singular forms “a”, “an”, and “the” as used herein include plural referents unless the context clearly dictates otherwise. Thus, for example, reference to “a lipophilic compound” includes reference to one or more of such compounds.

The term “container” as used herein refers to an enclosure suitable for storing the mixture protected from oxygen and moisture. It can be easily opened by the end user and emptied right before consumption. Optionally the container allows for preparation of the phospholipid dispersion by injecting heated and pressurized liquid into the container that holds the mixture. In that situation the container is designed as a cartridge compatible for use in available hot beverage preparation machines, in particular machines for single-serve coffee capsules.

The term “natural” refers to components derived from plant or animal sources. The term includes derivatives of components derived from plant or animal sources.

The term “nature-derived” refers to components that are constructed by linking molecular building blocks together that are found in nature, either in pure form or obtained by enzymatic treatment of natural substances.

The term “dispersion” as used herein refers to a mixture of two or more substances that are not soluble in each other and wherein one is present as dispersed droplets, particles, aggregates, colloids, or vesicles in the other substance that is then present in the form of a liquid. Before preparation of a beverage by mixing the contents of the container with water or water-based liquids, the contents of the container is generally present as mixture of an oil phase comprising the phospholipids and other oil-soluble ingredients optionally encompassing poorly water-soluble solids, mixed in a hydrophilic polyol solvent phase in which additional ingredients may be dissolved.

The mixture preferably is a paste, liquid, viscous liquid, a semi-solid or a solid. The mixture is preferably in the form of a solution, or dispersion, or a combination thereof. In one embodiment it is comprised of a fully dissolved mixture of the one or more lipophilic or poorly water-soluble bioactive ingredients, the one or more phospholipids, the one or more oils and/or triglycerides and the one or more liquid or solid polyols, polyol sugars, natural sugars, monohydric and/or polyhydric alcohols. In another embodiment it is comprised of a fully dissolved mixture in oil dispersed in the one or more liquid or solid polyols, polyol sugars, natural sugars, monohydric and/or polyhydric alcohols. Preferably, at least part of said one or more phospholipids are dispersed in said one or more liquid or solid polyols, polyol sugars, natural sugars, monohydric and/or polyhydric alcohols, the dispersion being a suspension, a colloidal mixture and/or an emulsion. The mixture can also be a mixture of a solution and a suspension, a colloidal mixture and/or an emulsion. Hence, in a preferred embodiment, the mixture is a suspension, a colloidal mixture and/or an emulsion or a combination of a solution and a suspension, a colloidal mixture and/or an emulsion. It is further preferred that less than 25% of the phospholipids are present in the mixture in the form of liposomes with a mean particle diameter of less than 1 μm, preferably less than 20%, more preferably less than 15%, more preferably less than 10%, more preferably less than 5%. I.e., preferably 75% or more of the one or more phospholipids is present in the form of a suspension, colloidal mixture and/or emulsion, preferably a suspension or colloidal mixture, preferably 80% or more, preferably 85% or more, more preferably 90% or more, more preferably 95% or more of the one or more phospholipids is present in the form of a suspension, colloidal mixture or emulsion, preferably a suspension or colloidal mixture. The mixture is not a solubilisate.

After exposing this mixture to and mixing it with water, or water-based liquids at elevated temperature the formulation is largely present in the form of a phospholipid dispersion, which is preferably homogeneous. Preferably the phospholipid dispersion is a colloidal phospholipid dispersion.

The term “stable” and “homogeneous” as used herein refer to the beverage remaining fully dispersed without visible large scale flocculation, aggregation, precipitation, frothing or phase separation when kept, without agitation, for at least half an hour after preparation.

The term “viscous” is used herein according to its art-recognized meaning and can be read as having a viscosity higher than that of water at the same temperature.

The terms “biologically active” and “bioactive” are used interchangeably herein. The terms refer to any natural, nature-derived or synthetic material that causes a biological response in a living tissue or cell.

The term “paste” as used herein refers to a material that behaves as a soft solid with a firm consistency and without the clear properties of a liquid.

The term “water-free” as used herein means containing less than 10% water on a weight basis, preferably less than 7.5 wt % water.

The term “HLB” as used herein refers to a hydrophilic-lipophilic balance of a surface-active material (e.g. surfactant, emulsifier). An HLB value of 0 corresponds to a completely hydrophobic (i.e., lipophilic) molecule, and a value of 20 corresponds to a completely hydrophilic (i.e., lipophobic) molecule. A “high HLB” refers to an HLB of at least 10. A “low HLB” refers to a HLB of at most 10.

The key finding underlying the present invention, which is necessary to enable the manufacturing of, and also the eventual processing in a common household situation of, a concentrated water-free (phospho) lipid premix capable of containing a range of bioactive substances, is that the premix formulation must contain a certain percentage of negatively charged phospholipids and/or phospholipids that can become negatively charged or become increasingly negatively charged by adding to the mixture—or processing the mixture beforehand with—a weak basic substance, e.g. a salt such as sodium bicarbonate. Such percentage of negatively charged phospholipids, when present in the mixture at the moment of dispersion in hot water or water-based liquid, greatly and critically improves the dispersion capacity of the phospholipids, taking away the need for advanced laboratory high-shear homogenization equipment and/or the addition of synthetic high HLB surfactants to obtain a homogeneous, colloidal phospholipid dispersion, but instead allowing it to be made by simply exposing it to and mixing it with heated (tap) water or another hot aqueous beverage, such as milk or juice.

The phospholipid fraction preferably constitutes between 5% and 40% of the total mixture by weight. The weight ratio of the phospholipid fraction to the polyol, polyol sugars, natural sugars, monohydric and/or polyhydric alcohol or the mixture thereof is preferably between 1:7.5 and 2:1. In another preferred embodiment, the phospholipid fraction preferably constitutes between 10% and 40% of the total mixture by weight and the weight ratio of the phospholipid fraction to the polyol, polyol sugars, natural sugars, monohydric and/or polyhydric alcohol or the mixture thereof is between 1:5 and 2:1. The phospholipid fraction can be a single phospholipid or a mixture of different phospholipids. Phospholipids that can be used in this invention are phospholipids known in the art for the production of liposomes, in particular amphipathic negatively charged or non-charged vesicle-forming phospholipids. The phospholipids can comprise saturated or unsaturated fatty acid side chains and feature a polar headgroup based on choline, glycerol, ethanolamine or phosphatidic acid. Alternative phospholipids of similar amphipathic vesicle-forming nature can also be included in the formulation. Non-limiting examples of suitable natural phospholipids are fat-free soy lecithin, sunflower lecithin, rapeseed lecithin egg yolk lecithin, cow milk lecithin and krill lecithin. Hydrogenated or partially hydrogenated versions thereof and synthetic phospholipids such as dimyristoyl phosphatidyl choline, dipalmitoyl phosphatidyl choline and distearoyl phosphatidyl choline can be added to the phospholipid mixture.

It is a key aspect of this invention that a at least 2% and preferably at least 5% on a weight basis of the phospholipid fraction in the mixture bears a net negative charge or is deprotonable. In the latter case, the mixture further comprises a weak basic salt. Preferably not more than 50% of the phospholipids is present in negatively charged or deprotonable form. ‘Bearing a net negative charge’ as used herein means that these phospholipids have more (weakly) acidic groups than (weakly) basic groups in their molecular structure and that they are formulated as anions with a counter (cat) ion that can be sodium, potassium or ammonium. ‘Deprotonable’ as used herein is defined as having (weakly) acidic groups with a pKa in water of below 9 and losing one or more protons upon exposure to (weak) bases with a pKb in water of below 9. These (weakly) acidic phospholipids are deprotonated by the preferably weak basic salts present in the mixture after exposure to and mixing with warm water or a water-based liquid during preparation. Phospholipids bearing a net negative charge that are available and included in the mixture in anionic form, or that are obtained after pre-treatment of weak acidic phospholipids with (weak) basic salts while preparing the mixture are for instance the sodium, potassium and other e.g., ammonium salt forms of phosphatidylglycerol, phosphatidylinositol, phosphatidylserine and phosphatidic acid. Examples of phospholipids that are obtained by enzymatically combining natural or nature-derived molecular building blocks are sodium dipalmitoyl or distearoyl phosphatidylglycerol.

In a preferred embodiment the one or more phospholipids, whether or not deprotonated and negatively charged or deprotonable, are natural—as present in natural phospholipid fractions. Natural deprotonated negatively charged or deprotonable phospholipids are relatively low in pure phosphatidylcholine but are relatively high in lecithin fractions that are less pure in terms of phosphatidylcholine content, containing higher percentages of lysophospholipids, glycolipids, phosphatidylethanolamine, phosphatidylserine, phosphatidic acid, phosphatidylglycerol or phosphatidylinositol or combinations thereof. Examples of such lecithin fractions rich in deprotonated or deprotonable natural phospholipids are soy lecithin, sunflower lecithin, rapeseed lecithin and egg lecithin with a phosphatidylcholine purity grade below 50%. The anionic phospholipids such as phosphatidic acid, phosphatidylglycerol, phosphatidylserine and phosphatidylinositol may be present already in anionic form or can be (further) deprotonated by adding weak basic salts that have a pKb of 9 or lower when the premix is prepared. These salts may also be just added to the mixture anticipating the deprotonation to occur when the mixture is exposed to and mixed with warm water or a water-based liquid. A preferred weak basic salt is sodium or potassium bicarbonate or any other salt featuring a weak basic anion. Also carbonate, hydrogen phosphate and dihydrogen phosphate can be used.

The one or more phospholipids are preferably natural or nature-derived. In a preferred embodiment all phospholipids, oils, polyols, polyol sugars, natural sugars, monohydric and/or polyhydric alcohols, optional surfactants and actives are natural or nature-derived.

Salts may be present in the mixture in the container after preparation. Weak basic salts present in the mixture may be leftovers after the deprotonation of phospholipids during the preparation of the mixture but may also be present anticipating deprotonation of phospholipids upon preparation when the mixture is exposed to and mixed with warm water or a water-based liquid. Hence, if the at least 2 wt % of the phospholipids that bears a net negative charge and/or is deprotonable comprises deprotonable phospholipids, optionally in combination with phospholipids that bear a net negative charge, the mixture further comprises a weak basic salt. If the at least 2 wt % of the phospholipids consists of phospholipids bearing a net negative charge and phospholipids in deprotonable form do not form part of said at least 2 wt % of the phospholipids, the weak basic salt may be absent. Weak basic salts with the aim to deprotonate phospholipids in the mixture are preferably added in a quantity between 0.2 mmol and 2 mmol per gram phospholipid present in the mixture. A preferred weak basic salt is sodium or potassium bicarbonate or any other salt comprising a weak basic anion. Of these carbonate, citrate, hydrogen citrate, tartrate, malate, glutarate, ascorbate, hydrogen phosphate and dihydrogen phosphate are preferred.

Additional salt can be present to improve the taste and texture of the final product and/or be added as an additional bioactive ingredient.

The polyol, polyol sugars, natural sugars, monohydric and/or polyhydric alcohol phase constitutes between 10% and 75% of the total mixture by weight. In some embodiments, the polyol, polyol sugars, natural sugars, monohydric and/or polyhydric alcohol phase constitutes between 10% and 50% of the total mixture by weight. In one preferred embodiment, the mixture comprises one or more liquid polyols, monohydric and/or polyhydric alcohols. The polyol, polyol sugars, natural sugars, monohydric and/or polyhydric alcohols or mixtures thereof, or polyols, monohydric and/or polyhydric alcohols, have an aliphatic structure with a melting point of the mixture below 80 degrees Celsius. Preferred examples of monohydric alcohols are ethanol, 2-propanol and tertiary butanol. Preferred examples of polyhydric alcohols are propylene glycol, 1,3 propane diol, glycerol, the several isomers that are available of butylene glycol, erythritol, tagatose and other polyol sugar alcohols. An even more preferred example of the polyol is glycerol, optionally mixed with one or more polyol sugar alcohols. Optionally, natural sugars can be added to the mixture with sucrose, glucose, fructose, lactose, maltose and galactose as preferred examples. Optionally a low percentage of water is allowed in this polyol solvent mixture. Optionally a low percentage of inulin or similar fructans from the class of dietary fibre polysaccharides can be added.

The bioactive ingredient is a lipophilic substance or a substance that is poorly soluble in water at room temperature, while it is well soluble or solubilizable in the oil phase, the monohydric and/or polyhydric alcohol (polyol) phase, or in the mixture of oil, the polyol, the phospholipids and optionally the additional surfactants. Poorly soluble in water is defined here as 1 gram of solute requiring more than 30 mL of water to be dissolved. With the term “lipophilic” is meant a log octanol/water value (log P value) of at least 0. The mixture may comprise more than one biologically active ingredient. The oil itself may be a bioactive substance, or the bioactive substance may constitute (part of) the oil phase. It is also possible that the phospholipids have bioactivity. The total amount of bioactive ingredients present in the mixture is preferably at least 0.5 weight % and preferably below 20 weight % of the total mixture. Preferably, the total amount of lipophilic bioactive ingredients present in the mixture is 1-20 weight % of the total mixture, more preferably 2-20 weight %, such as 3-15 weight %, 4-15 weight % or 4-10 weight %. For instance, about 1%, about 2% or about 5% of the lipophilic bioactive ingredients present in the mixture.

The bioactive ingredients are preferably present in an amount sufficient to exert their bioactive effect.

In preferred embodiments the bioactive ingredient is a lipophilic vitamin or provitamin, a polyphenol or flavonoid substance, a endocannabinoid structure, a cannabinoid compound, a fatty acid neurotransmitter, an etheric or essential oil, a terpene derivative, or a saturated or polyunsaturated fatty acid or its chemical derivative, such as an ester or salt thereof, or combinations thereof.

In specific embodiments the active ingredient is a endocannabinoid substance from the N-acyl ethanolamine (NAEs) class, or the monoacylglycerol class or mixtures thereof.

In specific embodiments the active ingredient is an aliphatic (poly) alcohol ester or amide of a polyunsaturated fatty acid (omega 3, 6 or 9), or mixtures thereof.

In specific embodiments the active ingredient is a cannabinoid substance.

In specific embodiments the active ingredient is a hydrophobic vitamin, a hydrophobic provitamin, or a hydrophobic derivative of a hydrophilic vitamin.

In specific embodiments the active ingredient is a polyphenol substance from the group of plant flavonoids, stilbenoids, phenylethyl acids or their derivatives, or mixtures thereof.

In specific embodiments the active ingredient is an essential oil from with a chemical structure based on plant terpenes or terpenoids.

Examples of cannabinoids are: THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran).

Examples of vitamins and provitamins: vitamin A variants, derivatives and provitamins (including beta-carotene), the different vitamins B and derivatives, vitamin C and derivatives, vitamin D variants, vitamin E variants, vitamin K1 and K2 and derivatives, coenzyme Q10 and related quinones, NADH and NADP and variants.

### Examples of Plant Polyphenol Classes are

Flavonoids including their glycoside derivatives: Flavones (Apigenin, Luteolin), Flavonols (Isorhamnetin, Kaempferol, Myricetin, Fisetin), Quercetin, Flavanones (Hesperetin, Naringenin) Flavanols and their polymers (Catechin, Gallocatechin and their corresponding gallate esters Epicatechin, Epigallocatechin and their corresponding gallate esters, Theaflavin its gallate esters), Isoflavones (Genistein, Glycitein), Stilbenoids (Resveratrol, Pterostilbene), Anthocyanins (Cyanidin, Malvidin), Phenolic acids and their esters (Caffeic acid and esters, Cinnamic acid and esters, Ellagic acid and esters, Ferulic acid and esters, Gallic acid and esters), Salicylic acid and esters, Curcumin and related compounds, Flavonolignans (silymarin), Eugenol, Capsaicin, Alpha-lipoic acid, Apocynin, Tyrosol, Hydroxytyrosol and Oleuropein.

Examples of carotenoid terpenoids are: Alpha-carotene, Astaxanthin, Beta-carotene, Canthaxanthin, Cryptoxanthin, Lutein, Lycopene, Zeaxanthin

Examples of essential oil components from plants with monoterpene, diterpene or sequiterpene chemical structures: including but not limited to carvacrol, eugenol, nerolidol, thymol, beta-caryophyllene, humulene, linalool, menthol, geraniol, borneol, sabinene.

The oil fraction comprising natural oils and/or triglycerides constitutes between 5% and 40% of the total mixture by weight. In some embodiments, the oil fraction comprising natural oils and/or triglycerides constitutes between 10% and 40% of the total mixture by weight. The weight ratio of this fraction to the phospholipid fraction is preferably between 1:2 and 1:0.5. The oil, and/or triglyceride fraction can be a single vegetable oil or a mixture of different natural oils and/or triglycerides. Preferred (natural) oils are ester derivatives of fatty acids or fatty alcohols. Of the fatty acid esters especially triglycerides are preferred. Some triglycerides have bioactivity and thus complement the health benefit profile of the formulation. Examples of triglycerides are vegetable oils from coconut, cocoa beans, babassu nuts, avocado, soy, sunflower, olives, walnuts, macadamia nuts, hempseed, rosehip seed, pomegranate seed, linseed, camellia (tea) seed, sea buckthorn seed, raspberry seed and other berry seeds that are known to have health promoting effects. A preferred triglyceride is fractionated coconut oil, especially the fractions high in caprylic acid (C8), also referred to as medium chain triglycerides (MCT). Special mention is made here of triglycerides and ethyl esters of the bioactive omega-3 fatty acids docosahexaenoic acid (DHA) and eicosapentaenoic acid (EPA) that can be present in high percentages in certain algal oils.

As indicated herein above, there is no need for high HLB synthetic surfactants in the mixture of the present invention. However, one or more surfactants may be present. Therefore, in one embodiment, the mixture comprises one or more additional surfactants. The one or more additional surfactants preferably are of natural origin or are derivatives of bioactive substances that are found in nature. In a preferred embodiment, the one or more additional surfactants are deprotonable in the presence of weak basic salts, acquiring a negative charge, which increases their dispersion capacity, ascorbyl palmitate being a prime example. In another embodiment the one or more additional surfactant is lysophosphatidylcholine. Other examples include citric acid esters of mono- and diglycerides of fatty acids, such as E472c.

Optionally synthetic surfactants can be added but preferably not more than 10% on a weight basis of the total mixture.

If present, the one or more additional surfactants is preferably present in a weight ratio to the one or more phospholipids in the mixture of between 1:2 and 1:20.

The mixture may further comprise ingredients that enhance taste, colour, viscosity, smell or chemical stability of the ingredients. Examples of additional ingredients are sweeteners including sugars or polyols, salts, minerals, colouring agents, etheric oils or other scent and taste enhancers, including coffee, milk powder, cocoa, tea, ginger, etc. Preferred mixtures contain tea powder, milk powder and/or cocoa powder between 5% and 20% by weight of the total formulation.

The mixture is preferably composed of only natural or nature-derived components.

Besides poorly water-soluble and lipophilic biologically active substances the mixture can also comprise hydrophilic active substances in quantities that amount to their recommended daily dose or the dose that is known to lead to the desired biological effect in the body. Examples of these actives are water-soluble vitamins, notably the vitamins B and vitamin C, minerals, water-soluble coenzyme factors, nucleic acid derivatives and water-soluble amino acids and derivatives. Also known water-soluble small-molecular drugs can be added.

In a preferred embodiment the mixture contains 5-40 wt % of a phospholipid component comprising phosphatidyl choline and one or more negatively charged phospholipids selected from the group consisting of phosphatidic acid, phosphatidylserine, phosphatidylinositol, phosphatidyl glycerol and combinations thereof, wherein said negatively charged phospholipids forms a fraction of between 2% an 50% on a weight basis of said phospholipid component; 10-50 wt % of a polyol solvent- and 10-40 wt % of a lipophilic component selected from the group of natural triglyceride oils and one or more natural or nature-derived bioactive substances. In a further preferred embodiment the mixture contains 10-40 wt % of said phospholipid component.

The mixture for filling into the air- or oxygen-impermeable and liquid-impermeable container is preferably prepared by first mixing the one or more, preferably natural, phospholipids, wherein at least 2 weight % of the phospholipids is deprotonable and the other lipophilic ingredients with the oils, butters and/or triglycerides after which the polyol solvent mixture with the weak basic salt dissolved in it is added under moderate heating and mixing, thereby providing the deprotonated phospholipids. After the deprotonation step is completed other biological actives may be added that cannot be dissolved in the lipid mixture or the polyol phase beforehand. Also additional salts and other food ingredients, such as milk powder may be added at this stage and mixed into the formulation. All ingredients are preferably added and mixed under moderate heating, preferably not exceeding 80 degrees Celsius. The final step is to fill this mixture into the air- or oxygen-impermeable and liquid-impermeable containers, also preferably under moderate heating to allow for sufficient flowability of the mixture during filling. Other ways to prepare the mixture are also possible as is known to a person skilled in the art.

Optionally the mixture, when encapsulated in a container, is placed in the container under a protective atmosphere of inert gases, examples of which are nitrogen and argon. To further improve stability the mixture is optionally pretreated with inert gases or mixtures thereof during mixing, optionally to the extent the resulting mixture gets a foamy texture.

The preparation of the final drink is achieved by the end user by emptying the container into a cup or glass with heated water above 80 degrees Celsius, or by pouring hot water or water-based liquid onto the content of the container transferred to an empty cup or glass, and stirring the obtained mixture briefly and vigorously during 10 seconds, waiting a minute and mixing again, after which the drink is allowed to stand for another 3-5 minutes and is again mixed to achieve the homogeneous phospholipid dispersion that is ready to be consumed.

Alternatively the drink is obtained by processing the container as a cartridge in a coffee extraction machine as further described below.

The container can be any air- and moisture-tight flask, bottle, bag, tube, jar, sachet or cartridge that can be stored without allowing air into the interior of the container. The container can contain multiple dosing units in the form of a squeezable tube or as a bottle or flask with a dispensing pump, whereby a single dosing unit is preferably between 3 and 20 grams of the mixture. One embodiment features an apparatus with more than one dispenser placed next to each other, each dispensing a different mixture and producing a different drink, or in which the contents of different dispensers can be combined to form one specific mix drink. In other embodiments the container is designed to hold a single dose and are completely emptied to prepare one drink. These containers are preferably sachets or single-use cartridges that fit into dispensing machines or hot water extraction machines, such as coffee machines. Such container contains between 3 and 20 grams of the mixture in preferred embodiments. The material the containers are made of can be plastic, aluminum, or organic material that is air- and moisture tight. Preferred containers are biodegradable or recyclable.

The formulation can be included in a cartridge that is compatible with coffee extraction machines that are present on the market. Examples of suitable cartridges are described in, and demonstrated in the drawings of U.S. Pat. Nos. 5,897,899 A, 7,490,542 B2, WO 2004/064584 A1, EP 2 611 713 B1, WO 2012/037409 A1, U.S. Pat. No. 6,740,345 B2 and WO 2005/092160 A1, which are incorporated herein by reference.

There are no particular requirements for filling the cartridge or capsule with the mixture or components thereof. For instance, first, a mixture of all ingredients can be made, which is subsequently added to the cartridge. Alternatively, the components are separately added to the cartridge. Combinations are also possible, separate components and/or mixtures of two or more components are added separately to the cartridge. The mixtures of all or part of the components can be prepared at room temperature or at elevated temperature, such as at a temperature of between 2° and 80° C.

These cartridges can be cylindrically shaped or flat spheroids as is needed to fit in their respective machines. The cartridges are airtight and pierced during processing to force hot water through and form the required stable dispersion containing beverage upon extraction, which is collected in e.g. a cup to be orally administered as a beverage.

Extraction can be performed with any apparatus that is commercially available and intended for preparing warm or hot beverage by extracting ingredients, e.g. coffee, from cartridges. Examples are the coffee extraction machines from Nespresso, Lavazza, Illy, Dolce Gusto, Keurig and Philips, Said aqueous liquid is preferably water. One full extraction is preferably achieved with a water volume of between 50 and 200 mL of water.

A person skilled in the art will understand that the cartridge and apparatus should be compatible, i.e. an apparatus should be used for extraction to prepare the phospholipid dispersion that is compatible with the specific cartridge that contains the formulation and that allows extraction of the phospholipid dispersion from the specific cartridge.

The phospholipid dispersion of the bioactive ingredient(s) that is formed upon extraction and collected for consumption is physically and chemically stable during at least one hour after preparation. With physical stability is meant that the formulation is homogeneous, essentially without large-scale visual inhomogeneities such as precipitates, aggregates, clumps or phase separation. This degree of fine dispersion is needed to ensure maximal bioavailability of the encapsulated bioactive ingredient(s) after oral administration.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: Photographs of beverages prepared in Example 1A (left) and Example 1B (right).

### EXAMPLES

### Example 1A

Preparation of a Mixture that Fails to Produce a Fine Colloidal Phospholipid Dispersion Upon Mixing with Hot Water

This experiment shows that production of a homogeneous and fine phospholipid dispersion is not possible with a premix based on phospholipids that have not been treated with weakly basic salts during preparation of the premix to produce sufficient phospholipids bearing a net negative charge and to which no additional net negatively charged lipids were added.

The following container with mixture was prepared:
Aluminum foil sachet
With a mixture of
   2.0 gram soy phosphatidyl choline P30 (Lipoid GmbH, Germany)
   1.0 gram MCT oil (De Notenshop, Netherlands)
   0.3 gram tocopherol (Nutrilo GmbH, Germany)
   0.2 gram ascorbyl palmitate (Dr Behr GmbH, Germany)
   2.0 gram glycerol (De Tuinen BV, Netherlands)

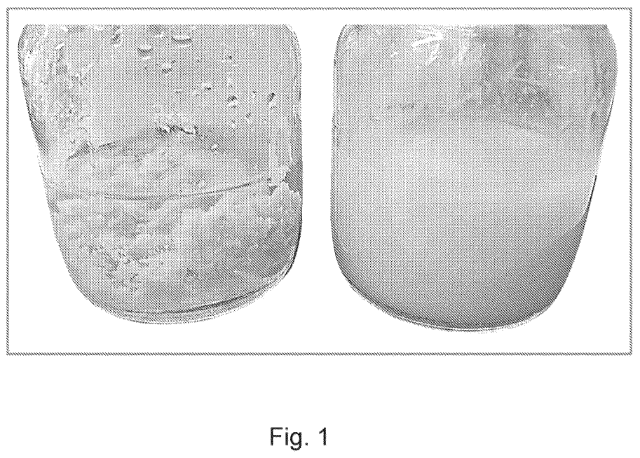

All ingredients except for glycerol were weighed, heated and mixed at about 80 degrees Celsius until fully dissolved. Onto the resulting mixture heated glycerol was carefully poured after which both phases were mixed thoroughly, placed in the container and cooled. The container was then emptied in 100 mL of water of 90 degrees Celsius and mixed, directly, after 30 seconds and after 5 minutes. However, after preparation the resulting lipid dispersion very quickly flocculated, formed white inhomogeneities and precipitates in the whole volume of the formulation (FIG. 1).

### Example 1B

### Preparation of the Same Mixture as in Example 1A but then with Sodium Bicarbonate to Deprotonate the Fraction of Deprotonable Phospholipids in the Mixture

The following container with mixture was prepared:
Aluminum foil sachet
With a mixture of
   2.0 gram soy phosphatidyl choline P30 (Lipoid GmbH, Germany)
   1.0 gram MCT oil (De Notenshop, Netherlands)
   0.3 gram tocopherol (Nutrilo GmbH, Germany)
   0.2 gram ascorbyl palmitate (Dr Behr GmbH, Germany)
   2.0 gram glycerol (De Tuinen BV, Netherlands) containing:
   60 mg of sodium bicarbonate (Carl Roth GmbH, Germany)

All ingredients except for glycerol and sodium bicarbonate were weighed, heated and mixed at about 80 degrees Celsius until fully dissolved. In 10 gram of glycerol 0.4 gram of sodium bicarbonate was dissolved by heating glycerol to 120 degrees Celsius and under stirring. Onto the lipid mixture 1.5 grams of the bicarbonate-containing glycerol was carefully poured after which both phases were mixed thoroughly. Upon mixing and heating foam formed that was allowed to dissipate. The resulting mixture was placed in the container and further cooled. The container was then emptied in 100 ml of water of 90 degrees Celsius and mixed. After 1 minute of intermittent stirring a fine dispersion started to form that took another 3 minutes of waiting and stirring to be completely homogeneous (FIG. 1).

### Example 2

### A Sachet Containing a Preparation of the Natural Active Ingredient Palmitoylethanolamide (PEA) Based on the Technology as Described in this Patent

The following container with mixture was prepared:
Aluminum foil sachet
With a mixture of
   1 gram soy phosphatidyl choline P30 (Lipoid GmbH, Germany)
   0.8 gram MCT oil (De Notenshop BV, Netherlands)
   0.2 gram cocoa butter (De Notenshop BV, Netherlands)
   0.6 gram palmitoylethanolamide (Ergomax BV, Netherlands)
   0.3 gram tocopherol ((Nutrilo GmbH, Germany)
   0.1 gram ascorbyl palmitate (Dr Behr GmbH, Germany)
   0.1 gram citrem (TER Ingredients GmbH&Co)
   2.6 gram glycerol (De Tuinen BV, Netherlands) containing:
   90 mg of sodium bicarbonate (Carl Roth GmbH, Germany)
   0.5 g of ginger liquid flavor enhancer (Pomona BV, Netherlands)

All ingredients except for glycerol and sodium bicarbonate were weighed, heated and mixed at about 90 degrees Celsius until fully dissolved. In 10 gram of glycerol 0.4 gram of sodium bicarbonate was dissolved by heating glycerol to 120 degrees Celsius and under stirring. Onto the lipid mixture 1.5 grams the bicarbonate-containing glycerol was carefully poured after which both phases were mixed thoroughly. Upon mixing and heating foam formed that was allowed to dissipate. The resulting mixture was placed in the container and further cooled. The container was then emptied in 100 mL of water of 90 degrees Celsius and mixed thoroughly. After 1 minute of intermittent stirring a fine dispersion started to form that took another 3 minutes of waiting and stirring to be completely homogeneous. The mixture remained stable for more than one hour.

### Example 3

### A Sachet Containing a Preparation of Cocoa Flavanols and DHA-Rich Algal Oil Based on the Technology as Described in this Patent

The following container with mixture was prepared:
Aluminum foil sachet
With a mixture of
   1.5 gram soy phosphatidyl choline P30 (Lipoid GmbH, Germany)
   0.5 gram DHA-rich algal oil (Progress Biotech BV, Netherlands)
   1.0 gram cocoa butter (De Notenshop BV, Netherlands)
   0.3 gram tocopherol (Nutrilo GmbH, Germany)
   0.2 gram ascorbyl palmitate (Dr Behr GmbH, Germany)
   1.5 gram glycerol (De Tuinen BV, Netherlands) containing:
   80 mg of sodium bicarbonate (Carl Roth GmbH, Germany)
   1.5 gram erythritol (Hhoya BV, Netherlands)
   50 mg xanthan gum (De Notenshop BV, Netherlands)
   0.2 gram cocoa flavanols (NanJing Manhay Medical Technology Co Ltd, China)
   0.5 gram dry milk powder (Campina BV, Netherlands)

All fats and oils were mixed with soy P30 and ascorbyl palmitate to form a fluid mixture under heating to 80 degrees Celsius. In 10 gram of glycerol 0.8 gram of sodium bicarbonate was dissolved by heating glycerol to 120 degrees celius and under stirring. After dissolution 10 grams of erythritol were added and melted-mixed into the glycerol phase until fully transparent. Onto the lipid mixture and under heating to 80 degrees Celsius 3 grams of the bicarbonate-containing glycerol-erythritol phase was carefully poured after which both phases were mixed thoroughly. Upon further mixing and heating foam formed that was allowed to dissipate. To this mixture the other ingredients were added (cocoa flavanols, cocoa powder, xanthan gum) and this mixture was heated again to 80 degrees Celsius and mixed thoroughly. The resulting mixture was placed in the container and further cooled. The container was then emptied in 100 ml of water of 90 degrees Celsius and mixed firmly. After 1 minute of intermittent stirring a fine dispersion started to form that took another 5 minutes of waiting and stirring to become completely homogeneous. The mixture remained stable for more than one hour.

### Example 4

### Capsule Containing Pomegranate Peel Extract and Pomegranate Seed Oil Based on the Technology as Described in this Patent

The following container with mixture was prepared:
Nespresso aluminum coffee cartridge 10 mL (iCafilas)
With a mixture of
   1.0 gram soy phosphatidyl choline P30 (Lipoid GmbH, Germany)
   0.8 gram MCT oil (coconut oil C8-C10 fraction) (De Notenshop BV, Netherlands)
   0.25 gram pomegranate seed oil (Natural Heroes BV, Netherlands)
   0.27 gram tocopherol (Nutrilo GmbH, Germany)
   0.2 gram ascorbyl palmitate (Dr Behr GmbH, Germany)
   0.2 gram lysophosphatidylcholine (Lipoid GmbH, Germany)
   2.5 gram glycerol (De Tuinen BV, Netherlands) containing:
   40 mg of sodium bicarbonate (Carl Roth GmbH, Germany)
   0.5 gram propylene glycol (Sanco Industries Inc, USA)
   0.2 gram pomegranate peel extract powder (NanJing Manhay Medical Technology Co Ltd, China)

All fats and oils were mixed with soy P30, lysophosphatidylcholine, ascorbyl palmitate and propylene glycol to form a fluid mixture under heating to 80 degrees Celsius. In 10 gram of glycerol 0.4 gram of sodium bicarbonate was dissolved by heating glycerol to 120 degrees Celsius and under stirring. Onto the lipid mixture and under heating to 80 degrees Celsius 1.5 grams of the bicarbonate-containing glycerol phase was carefully poured after which both phases were mixed thoroughly. Upon further mixing and heating foam formed that was allowed to dissipate. To this mixture the pomegranate peel extract was added and this mixture was heated again to 80 degrees Celsius and mixed thoroughly. The resulting mixture was poured into the aluminum coffee cartridge that was then sealed with aluminum foil in a special sealer device.

The cartridge with the mixture was then placed into a Nespresso coffee extractor machine and extracted with 100 mL warm water. The resulting phospholipid dispersion initially produced a white foam layer but quickly settled with a light pink color. It remained stable as a fine and clear dispersion for one hour.

### Example 5

### Capsule Containing Quercetin, MCT Oil and a Vitamin Mix Based on the Technology as Described in this Patent

The following container with mixture was prepared:
Nespresso aluminum coffee cartridge 10 mL (iCafilas)
With a mixture of
   1.0 gram soy phosphatidyl choline P30 (Lipoid GmbH, Germany)
   1.0 gram MCT oil (coconut oil C8-C10 fraction) (De Notenshop BV, Netherlands)
   0.27 gram tocopherol (Nutrilo GmbH, Germany)
   0.2 gram lysophosphatidylcholine (Lipoid GmbH, Germany)
   2.5 gram glycerol (De Tuinen BV, Netherlands) containing:
   61 mg of sodium bicarbonate (Carl Roth GmbH, Germany)
   0.5 gram propylene glycol with tangerine flavor (Pomona BV, Netherlands)
   0.2 gram quercetin (NanJing Manhay Medical Technology Co Ltd, China)
   0.26 gram sodium ascorbate (Van Beekum BV, Netherlands)
   0.16 gram zinc ascorbate (Parchem Inc, USA)

All fats and oils were mixed with soy P30, lysophosphatidylcholine, ascorbyl palmitate and propylene glycol to form a fluid mixture under heating to 80 degrees Celsius. In 10 gram of glycerol 0.25 gram of sodium bicarbonate was dissolved by heating glycerol to 120 degrees Celsius and under stirring. Onto the lipid mixture and under heating to 80 degrees Celsius 2.5 grams of the bicarbonate-containing glycerol phase was carefully poured after which both phases were mixed thoroughly. Upon further mixing and heating foam formed that was allowed to dissipate. To this mixture the quercetin and ascorbyl salts were added and this mixture was heated again to 80 degrees Celsius and mixed thoroughly. The resulting mixture was poured into the aluminum coffee cartridge that was then sealed with aluminum foil in a special sealer device.

The cartridge with the mixture was then placed into a Nespresso coffee extractor machine and extracted with 100 mL warm water. The resulting phospholipid dispersion was homogeneous and light yellow. It remained stable as a fine and clear dispersion for several hours.

### Example 6

### An Airtight Glass Jar Containing 10 Daily Doses of a Preparation of Cocoa Flavanols and DHA-Rich Algal Oil Based on the Technology as Described in this Patent

The following container with mixture was prepared:
Airtight closed glass jar of 100 mL volume
With a mixture of
   15 gram soy phosphatidyl choline P30 (Lipoid GmbH, Germany)
   5 gram DHA-rich algal oil (Progress Biotech BV, Netherlands)
   2 gram cocoa butter (De Notenshop BV, Netherlands)
   3 gram MCT (fractionated coconut oil) (De Notenshop BV, Netherlands)
   1.5 gram tocopherol (Nutrilo GmbH, Germany)
   0.6 gram ascorbyl palmitate (Dr Behr GmbH, Germany)
   0.35 gram citrem (TER Ingredients GmbH&Co)
   22.5 gram glycerol (De Tuinen BV, Netherlands) containing:
   540 mg of sodium bicarbonate (Carl Roth GmbH, Germany)
   10 gram erythritol (Hhoya BV, Netherlands)
   500 mg xanthan gum (De Notenshop BV, Netherlands)
   2 gram cocoa flavanols (NanJing Manhay Medical Technology Co Ltd, China)
   2 gram cocoa powder (De Notenshop BV, Netherlands)
   800 mg sodium chloride (De Notenshop BV, Netherlands)
   100 mg stevia extract (Hhoya BV, Netherlands)

All fats and oils apart from DHA-rich algal oil were mixed with soy P30 and ascorbyl palmitate to form a fluid mixture under heating to 80 degrees Celsius. In 10 gram of glycerol 0.8 gram of sodium bicarbonate was dissolved by heating glycerol to 120 degrees Celsius and under stirring. After dissolution 10 grams of erythritol were added and melted-mixed into the glycerol phase until fully transparent. Onto the lipid mixture and under heating to 80 degrees Celsius 3 grams of the bicarbonate-containing glycerol-erythritol phase was carefully poured after which both phases were mixed thoroughly. Upon further mixing and heating foam formed that was allowed to dissipate. To this mixture the other ingredients were added (cocoa flavanols, cocoa powder, xanthan gum) and this mixture was heated again to 80 degrees Celsius and mixed thoroughly. Upon further cooling to 50 degrees Celsius the DHA-rich algal oil was added to the mixture and thoroughly stirred. The resulting mixture was placed in the glass jar and further cooled. Upon taking one daily dose of around 7 grams and mixing this with 100 ml of water of 90 degrees Celsius followed by a few minutes of intermittent stirring a fine dispersion started to form that took another 5 minutes of waiting and stirring to become completely homogeneous. The mixture remained stable for more than one hour.

1. A sealed and air- or oxygen-impermeable and liquid-impermeable container, which encapsulates a mixture comprising:
one or more phospholipids, wherein at least 2 wt % of the phospholipids:
bears a net negative charge, and/or
is deprotonable, whereby the mixture comprises a weak basic salt,
one or more oils and/or triglycerides,
one or more lipophilic or poorly water-soluble biologically active ingredients, and
one or more liquid or solid polyols, polyol sugars, natural sugars, monohydric and/or polyhydric alcohols.

2. The container according to claim 1, wherein the container is a cartridge, an airtight sachet, bag, jar or an airless fluid dispenser.

3. The container according to claim 1, wherein all components of said enclosed mixtures are natural or nature-derived components and wherein the biologically active ingredients are natural and/or synthetic.

4. The container according to claim 1, wherein said one or more natural phospholipids are selected from the group consisting of soy, sunflower, linseed, rapeseed, egg, cow milk, krill lecithin and phospholipids and combinations thereof and wherein said one or more natural phospholipids have a purity grade in terms of phosphatidylcholine content of below 50%.

5. The container according to claim 1, wherein said negatively charged or deprotonable phospholipids are selected from the group consisting of phosphatidic acid, phosphatidylserine, phosphatidylglycerol, phosphatidylinositol and combinations thereof and/or their respective salts.

6. The container according to claim 1, wherein said mixture further comprises one or more weak basic salts.

7. The container according to claim 6, wherein said one or more salts are selected from the group consisting of a bicarbonate salt, a dihydrogen and monohydrogen phosphate salt and combinations thereof.

8. The container according to claim 1, wherein said one or more liquid monohydric and/or polyhydric alcohols are selected from the group consisting of glycerol, propylene glycol, sorbitol, erythritol, tagatose, xylitol, ethanol, poly(ethylene glycol) and combinations thereof.

9. The container according to claim 8, wherein said one or more liquid nontoxic monohydric and/or polyhydric alcohols comprises glycerol.

10. The container according to claim 1, wherein the biologically active ingredient is a lipophilic or poorly water-soluble biologically active ingredient.

11. The container according to claim 1, wherein the biologically active ingredient is a hydrophilic biologically active ingredient.

12. The container according to claim 1, wherein the mixture further comprises one or more additional surfactants of natural origin or nature-derived, besides the phospholipids

13. The container according to claim 1, wherein said one or more additional surfactants are selected from the group consisting of ascorbyl palmitate, lysophosphatidyl choline and combinations thereof.

14. The container according to claim 1, wherein the mixture comprises:
5-40 wt % of a phospholipid component comprising phosphatidyl choline and negatively charged or deprotonable phospholipids selected from the group consisting of phosphatidic acid, phosphatidyl ethanolamine, phosphatidyl glycerol, a glycophospholipid, and combinations thereof, wherein said negatively charged phospholipids forms at least 2 wt % of said phospholipid component,
20-50 wt % of a mixture of glycerol and liquid or solid polyols, polyol sugars or natural sugars
5-40 wt % of a lipophilic component selected from the group consisting of natural triglyceride oils
one or more natural plant bioactive substances.

15. A mixture comprising:
one or more phospholipids, wherein at least 2 wt % of the phospholipids:
bears a net negative charge, and/or
is deprotonable, whereby the mixture comprises a weak basic salt,
one or more oils, butters and/or triglycerides,
one or more lipophilic or poorly water-soluble biologically active ingredients, and
one or more liquid or solid polyols, polyol sugars, natural sugars, monohydric and/or polyhydric alcohols.

16. The mixture according to claim 15, which is a mixture as defined in claim 14.

17. The mixture according to claim 15 comprising:
lecithin or a mixture of more natural phospholipids in an amount equal to between 5 and 40 percent of the mixture on a weight basis
natural triglyceride oil or butter in an amount equal to between 5 and 40 percent of the mixture on a weight basis;
a mixture of one or more liquid or solid polyols, polyol sugars, natural sugars, monohydric and/or polyhydric alcohols in an amount equal to between 10 and 75 percent of the emulsion on a weight basis;
of which the lecithin/phospholipid fraction contains at least 2 weight % phospholipids bearing a net negative charge or which can be deprotonated or further deprotonated after addition of weak basic salt to the mixture.

18. A method for preparing the air- or oxygen-impermeable and liquid-impermeable container according to claim 1 comprising:
a) mixing one or more phospholipids, wherein at least 2 weight % of the phospholipids is deprotonable and the other lipophilic ingredients with the one or more oils, butters and/or triglycerides,
b) adding the one or more liquid or solid polyols, polyol sugars, natural sugars, monohydric and/or polyhydric alcohols with a weak basic salt dissolved in it to the mixture obtained in step a), whereby two phases are formed,
c) mixing both phases under moderate heating thereby creating and ensuring a sufficiently deprotonated phospholipid fraction,
d) optionally adding other biologically active ingredients that cannot be dissolved in the lipid mixture or the polyol phase beforehand to the mixture obtained in step c),
e) optionally adding and mixing salts, taste enhancers and other food ingredients, such as milk powder, into the mixture obtained in step d) under moderate heating, preferably not exceeding 80° C., and
f) filling an air- or oxygen-impermeable and liquid-impermeable container with the mixture obtained in step e).
